(19) United States
(12) Patent Application Publication
(10) Pub. No.: US 2024/0066261 A1
(43) Pub. Date: Feb. 29, 2024

(54) AN AUDIO DEVICE, METHOD AND SYSTEM FOR ALLEVIATING SYMPTOMS OF COLIC IN INFANTS

(71) Applicant: Colic Button AS, Hønefoss (NO)

(72) Inventor: Eleni Menti Larsen, HØNEFOSS (NO)

(21) Appl. No.: 18/261,257

(22) PCT Filed: Dec. 23, 2021
(86) PCT No.: PCT/NO2021/050283

§ 371 Date: Jul. 12, 2023

(30) Foreign Application Priority Data
Jan. 14, 2021 (NO) 20210050

(51) Int. Cl. A61M 21/02

(52) U.S. Cl. CPC A61M 21/02; A61M 2021/0027

(57) ABSTRACT

The disclosure relates to an audio device (10) for alleviating symptoms of colic in infants comprising: a loudspeaker (1), a controller (4) for controlling the loudspeaker, a sound input pattern to be fed to the loudspeaker, and a power source (5,7), wherein the audio device (10) is programmable to output audio sequences at two or more sound levels. The disclosure further relates to a method for alleviating symptoms of colic in infants and a system for alleviating symptoms of colic in infants.

### TECHNICAL FIELD

The present disclosure relates to an audio device for alleviating symptoms of colic in infants, a method for alleviating symptoms of colic in infants and a system for alleviating symptoms of colic in infants. More specifically, the disclosure relates to an audio device for alleviating symptoms of colic in infants, a method for alleviating symptoms of colic in infants and a system for alleviating symptoms of colic in infants and to as defined in the introductory parts of claim 1, claim 22 and claim 24.

### BACKGROUND ART

Colic is frequent, prolonged, and intense crying or fussiness in an apparently healthy infant. Colic can be particularly frustrating for parents because the baby's distress occurs for no apparent reason and no amount of consoling seems to bring any relief. These episodes often occur in the evening when parents themselves are often tired. Despite more than 50 years of research in this field, no one has been able to prove the link between stomach problems and infant colic. It is assumed that colic children are among those children who are unable to stop crying once they have started. The fact that colic usually ends at the age of three to four months, supports the theory that it is a developmental phenomenon and not a disease.

Colic is defined as intense and inconsolable crying and restlessness that lasts longer than 3 hours a day for more than 3 days of the week for more than 3 weeks. Episodes of colic usually peak when an infant is about 6 weeks old and decline significantly after 3 to 4 months of age. While the excessive crying will resolve with time, managing colic adds significant stress to caring for a newborn child.

Colic is stressful for parents. Research has shown an association between colic and the following problems with parent well-being:
Increased risk of postpartum depression in mothers.
Early cessation of breast-feeding.
Feelings of guilt, exhaustion, helplessness, or anger.
There is a high incidence peak of SBS (Shaken Baby Syndrome) in infants 2-4 months of age, just when colic is most prevalent.

There are well known and discussed calming remedies for colicky babies, such as:
carrying the infant
swaddling (it is believed that wrapping a blanket tight around the infant gives it the feeling of security)
smooth sounds, such as the hum from a vacuum cleaner or a dryer can be comforting to babies
playing soothing music, or repeating the “shh” sound to the little one
gentle movement and swinging
taking the infant for a car ride
using a pacifier

The problem with the solutions listed above is that these “magic bullet” remedies are unreliable, and only sporadically helps lessen the colic behavior.

There is thus a need for improved solutions to the above stated problem.

### SUMMARY

It is an object of the present disclosure to mitigate, alleviate or eliminate the above-identified deficiencies and disadvantages in the prior art and solve at least the above mentioned problem.

The present invention is based on the concept that random sound, commonly referred to as white noise when it extends over a wide frequency range or pink noise when it extends over the range of sound frequencies, has a calming effect on babies and young children. For example, it has been noticed that the most pronounced sound used to calm, or silence, children is “SHSHSH”, an oral simulation of random sound. Furthermore, it is believed that this is far better than the sound of the mother's heart beating or other quiet sounds such as birds chirping, and raindrops.

The present invention is not merely based on the recognition that white noise will have a calming effect on an infant. It is found that the use of at least two different sound patterns and/or sound levels, wherein an initial higher sound level followed by a lower one works well to change the crying habit of the infant. The higher sound level needs to be high enough to penetrate the sound of the infant's own crying and thus attracting the babies' attention.

As long as the sound is on, the infant stays calm, and most times falls asleep. The babies then tend to sleep longer. It is believed this happens because the sound stimulates the infant in a similar way to as if it is still in the mother's womb.

According to a first aspect of present invention there is provided an audio device for alleviating symptoms of colic in infants comprising: a loudspeaker, a controller for controlling the loudspeaker, a sound input pattern to be fed to the loudspeaker, and a power source, wherein the audio device is programmable to output audio sequences at two or more sound levels.

To help the infant get out of the crying, the initial higher sound level may be louder than the infants own crying. The infant then recognizes this sound and immediately calms down.

According to some embodiments, the loudspeaker is programmed to output a first sound pattern during a first time period at a first sound level, and automatically switch to output a second sound pattern during a second time period at a second sound level when the first time period has ended. The second sound level is lower than the first sound level.

A relatively short period of time is found sufficient for the first time period, before the switch to a lower sound level sufficient to keep the child's attention. This lower sound level will promote sleep or a state of calm for the infant.

According to some embodiments, the first time period is in the region 2-30 seconds, or 2-20 seconds, or 2-10 seconds, or 4-7 seconds, or 5 seconds, with a deviation of +/−0.5 seconds.

According to some embodiments, the second time period is indefinite.

A prolonged period of lower level sound is advantageous as the infant will be calmed, and the background sound will camouflage other ambient sound.

According to some embodiments the first sound pattern and second sound pattern is white noise of similar or different sound pattern/frequency mix.

It may be advantageous to use a sound pattern adapted individually to the infant.

According to some embodiments, the first sound level and/or the second sound level is adjustable.

Infants are individuals, and it may be advantageous to adapt sound levels to infants own crying volume and needs.

According to some embodiments, the audio device further comprises: a start button for starting or restarting the audio output sequence.

According to some embodiments, the audio device further comprises: a sound sensor, and a sound sensor analyzing module.

Thus, it is possible to monitor response and infant state/crying habits/level.

According to some embodiments, the sound sensor analyzing module is enabled to define the first sound level relative to the sensed and analyzed sound level from the sound sensor.

An adaptability to the individual infant is advantageous, as higher sound than necessary thus can be avoided.

According to some embodiments, the audio device further comprises: an automatic restart module for restarting the audio output sequence when the first time period has expired and sensed and analyzed sound level from the sound sensor is higher than the second sound level.

Thus, it may be possible to re-calm the infant if it's unexpectedly woken up by another disturbance or happening.

According to some embodiments, the audio device further comprises: a power storage device for powering the audio device.

According to some embodiments, the power storage device is one of, or a combination of: a one-time use battery, a rechargeable battery, a capacitor, a remote connected power source, or a power grid connector connecting the audio device to a power grid.

According to some embodiments, the audio device further comprises: a configurable audio sound pattern device providing the sound input pattern to be fed to the loudspeaker.

Thus, the device may be adapted to individual sound output requirements and wishes.

According to some embodiments, the configurable audio sound pattern device is a memory chip inserted in a memory chip reader comprised in the audio device.

Thus, changing or making individual sound patterns are more easily provided.

According to some embodiments, the audio device further comprise: a remote controller, a communication module for communicating with the remote controller, and an antenna for signal communication.

Thus, it is possible to facilitate remote monitoring and control.

According to some embodiments, the communication module comprise one or more of: Wi-Fi communication module, Bluetooth communication module, RFID communication module, NFC communication module, Radio Frequency communication module.

According to some embodiments, the remote controller is one or more of: a smart phone, a tablet, a computer, a cloud based service, or a dedicated communication device.

According to some embodiments, the audio device further comprise: a sound directing device for directing the sound output from the loudspeaker.

Thus, the sound may be shielded such that it is less recognizable for others than the infant to be calmed.

According to some embodiments, the audio device is comprised in an enclosure for being carried around the neck of a caretaker.

According to some embodiments, a necklace string with a length equal to minimum distance from loudspeaker to the nearest one of the infant's ears.

It may be advantageous to have a readily available distance measure, to avoid damaging the infants hearing.

According to some embodiments, the audio device is comprised in an enclosure for being mounted close to an infant resting pillow/headrest position.

According to a second aspect of present invention there is provided a method for alleviating symptoms of colic in infants, the method comprise to: provide an audio device wherein the audio device output a white noise for a first short period of time louder than the infant's crying sound level, when the duration of the first short period of time has expired outputting a second white noise at a lower sound level, or a different second noise pattern at a lower sound level, and keep the second sound level and pattern indefinite or until the infant wakes up.

According to some embodiments, the audio device is provided by an audio device according to the first aspect of the present invention.

According to a third aspect of present invention there is provided a system for alleviating symptoms of colic in infants, the system comprising: an audio device according the first aspect, and a remote processing device.

According to some embodiments, the system comprises: a cloud based service communicating one or more of the audio device, the remote controller, and the remote processing device.

Effects and features of the second and third aspects are to a large extent analogous to those described above in connection with the first aspect. Embodiments mentioned in relation to the first aspect are largely compatible with the second and third aspects.

The present disclosure will become apparent from the detailed description given below. The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### Terminology

The term “white noise” is to be interpreted as a signal having equal intensity at all frequencies audible by the human ear. In present invention it shall however also in some embodiments be understood that true white noise may be replaced by other frequency compositions, and also replaced by recorded audio sequences. Sound that extends over the range of sound frequencies which often is referred to as “pink noise”, “brown noise” or other may also fall in under the same definition.

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 shows a top view of the audio device according to an embodiment of the present disclosure.

FIG. 2 shows the internal layout of the device in FIG. 1.

FIG. 3 shows a first side view of the audio device according to the embodiment of the present disclosure as shown in FIG. 1.

FIG. 4 shows a second side view of the audio device according to the embodiment of the present disclosure as shown in FIG. 1.

FIGS. 5A and B shows two alternative sound level diagram of different embodiments of present invention.

FIG. 6 shows an example waveform of a Gaussian white noise signal plotted on a graph.

FIG. 7 shows a system overview of some elements provided in a variety of networked/cloud based system layout embodiments.

FIG. 8 shows a flow chart of one method embodiment of the present disclosure

FIG. 9 illustrates typical White and Pink noise

FIG. 10A shows sound measure at 50 cm from loudspeaker

FIG. 10B shows sound measure at 2.5 cm from loudspeaker

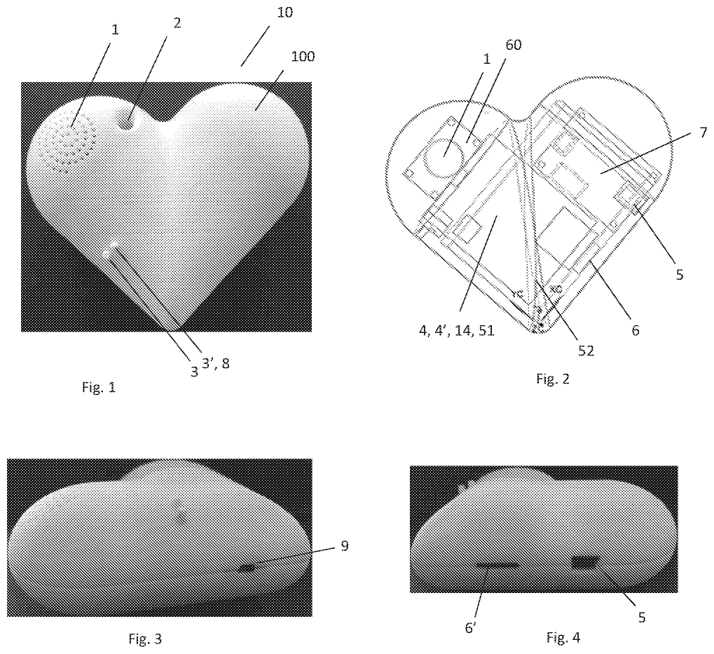

### DETAILED DESCRIPTION

The present disclosure will now be described with reference to the accompanying drawings, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

FIG. 1 shows a first example of present invention formed to be associated with well-being device.

The first aspect of this disclosure shows an audio device 10 for alleviating symptoms of colic in infants comprising: a loudspeaker 1, a controller 4 for controlling the loudspeaker 1, a sound input pattern to be fed to the loudspeaker, and a power source 5,7, wherein the audio device 10 is programmable to output audio sequences at two or more sound levels.

The sound audio device 10 may output a first audio sequence for getting the attention of an infant and play a second audio sequence, and optional further audio sequences, when attention is achieved.

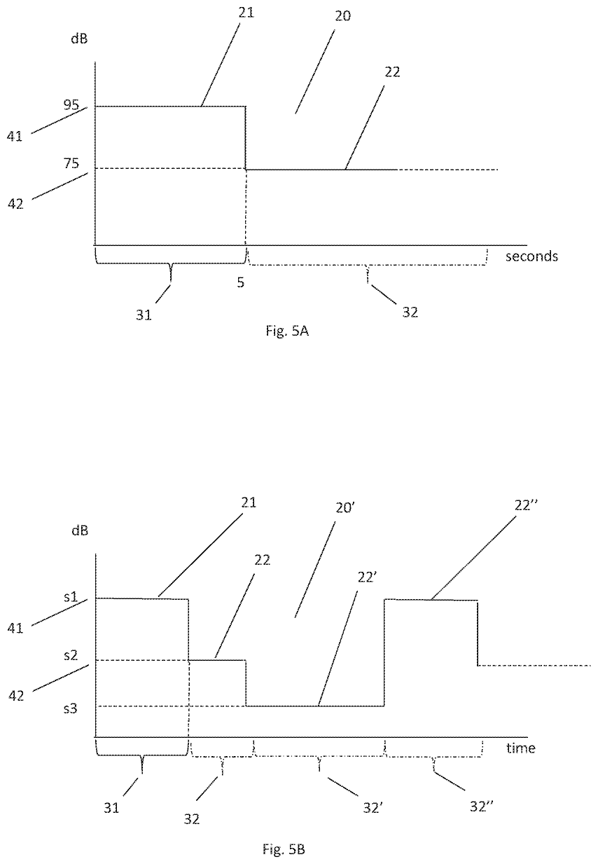

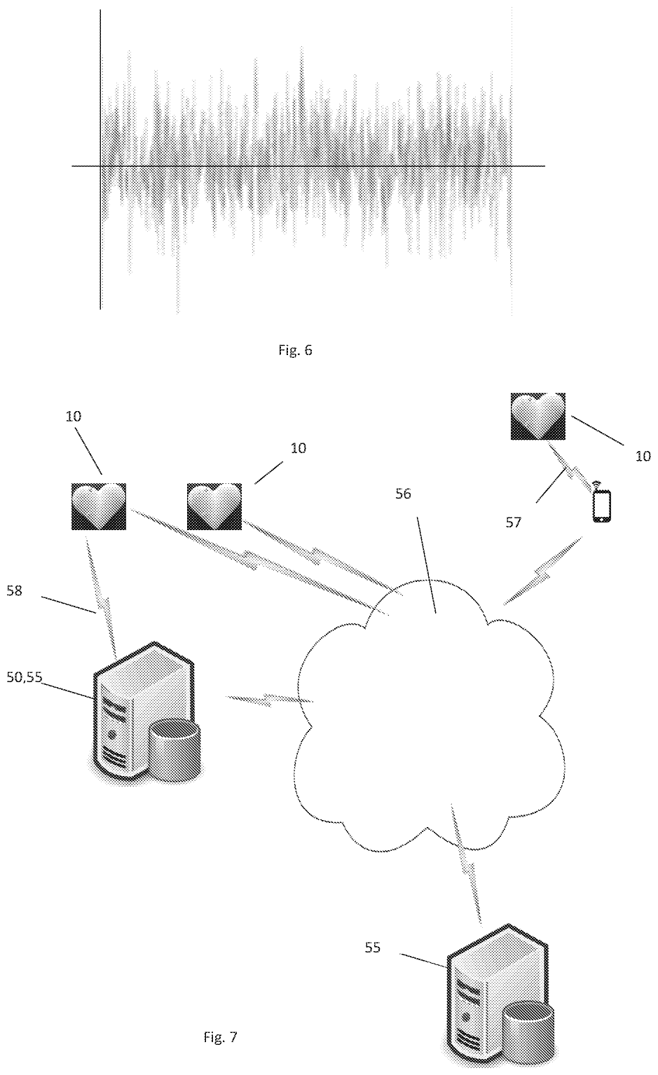

In a preferred embodiment of the invention the loudspeaker 1 is programmed to output a first sound pattern 21 during a first time period 31 at a first sound level 41, as illustrated in FIG. 5A. When the first time period has expired, the loudspeaker output automatically switch to output a second sound pattern 22 during a second time period 32 at a second sound level 42. When using the device to calm an infant, it is advantageous that the second sound level 42 is lower than the first sound level 41.

The aim for the duration of the first time period 31 is to attract attention of a crying infant and is advantageous in the region 2-30 seconds, or 2-20 seconds, or 2-10 seconds, or 4-7 seconds, or 5 seconds, with a deviation of +/−0.5 seconds. The first time period 31 outputting a sound at a first sound level 41 should be played until the infant stops crying, but not too long, in order to avoid any health issues, such as damage to the ears. The time period needed may vary from infant to infant and is from experience in the region of 5 to 15 seconds, but slightly longer periods may be needed. Also, shorter first time period may be adapted, although for practical reasons a standard 5 sec duration of the startup period may be chosen.

The objective for present invention is thus to provide a device for alleviating symptoms of colic in infants, a colic stopper, which helps infants suffering from colic out of their crying. The present invention helps infants with symptoms of colic to calm down enough so that they can be fed. The present invention also helps infants with symptoms of colic to calm down and/or fall asleep and sleep longer.

When the first time period 31 has expired, the audio device automatically regulates itself down to a second sound level 42 sufficient for calming the infant to a state of sleep or at least a state of prolonged calm. The sound levels necessary to be output depend on how close the infant is to the sound audio device 10. Nevertheless, the first sound level 41 should be sufficiently high for the infant to hear it through his own crying, and the second sound level 42 may be lower, but high enough to keep the infant at a state of prolonged calm. It is however anticipated that the second time period 32 is indefinite, or as long as the infant is expected to sleep/be calm.

Observations have shown that when the infant has a specific cause for crying—such as hunger or pain, which requires more specific attention, the effect of the audio device 10 will not override the infant's primary needs. The sound audio device 10 will then help the infant to calm down enough, such that it may be nappy changed or fed. If asleep under the second time period 32, the infant will wake up if hungry, in pain or otherwise uncomfortable/ill.

Furthermore, it is assumed that random sound with low volume is not harmful, equated with most “unprogrammed” sound—such as flow of water, wind through the trees, rain, etc.

In accordance with an embodiment of present invention the first sound pattern 21 and second sound pattern 22 is white noise of similar or different sound pattern/frequency mix.

It is believed that the sounds the child hears in the mother's womb are not the mother's heart, but the blood flowing through the body in a high volume of over 95 dB Swish sound. Infants cry at an estimated 90 dB and to help the child get over the crying, the first sound level 41 should be higher than 90 dB, or at least higher than the intensity of the infants crying. The aim is to simulate the white sound they recognize from life in the womb of the mother and they immediately become calm.

The present invention is in one advantageous embodiment implemented to output a first sound pattern 21 having equal intensity across all frequencies audible by the human ear (the traditional meaning of “white noise”), and then, when the first time period 31 has expired, a second sound pattern 22 having for example substantially equal power per octave (the traditional meaning of “pink noise”) is output. Non limiting examples of White noise and Pink noise are exemplified in FIG. 9. The second sound level 42 may also be chosen to be output at a considerably lower volume than the first sound level 41. Thus, the second sound pattern may be experienced as “softer” and more “pleasant” sound to the ear.

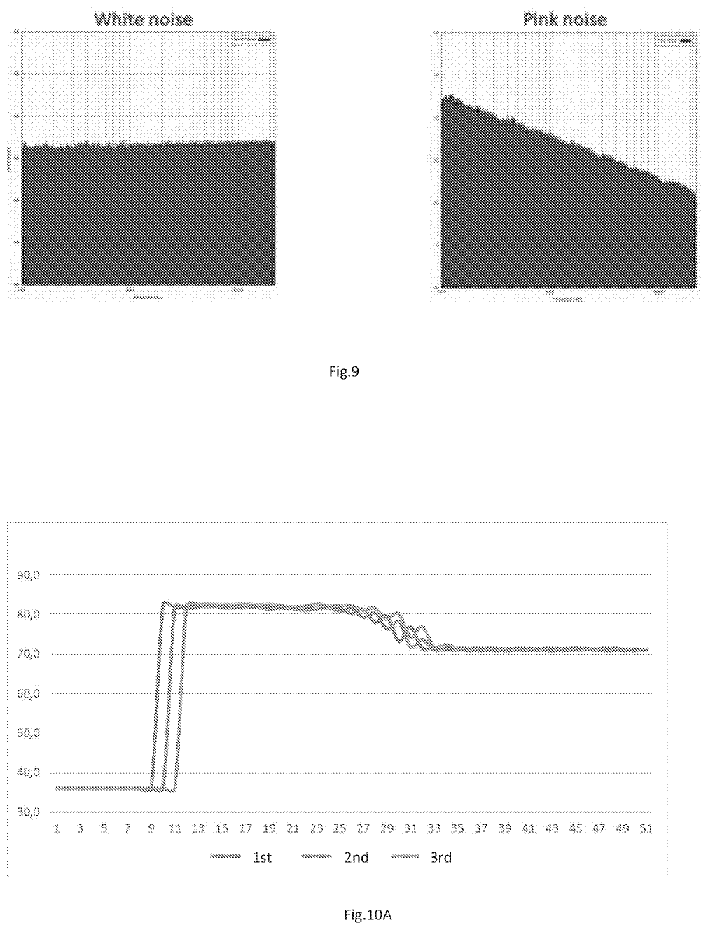

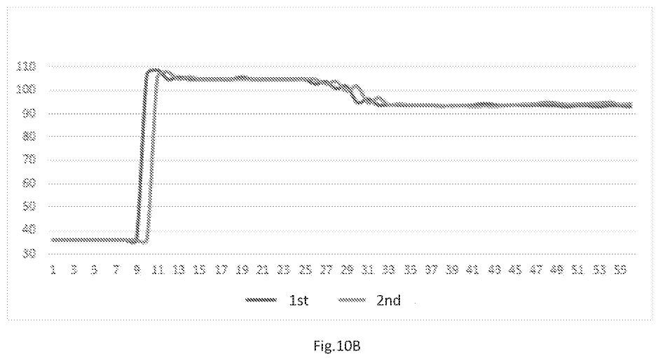

In FIG. 10A it is shown a measurement of the sound at 50 cm from the loudspeaker during 3 test runs, illustrating an embodiment of the present disclosure wherein a first sound level 41 is higher at an average of approximately 82 dB, and a second sound level 42 at approximately 71 dB.

In FIG. 10B it is shown a measurement of the sound at 2.5 cm from the loudspeaker during two test runs, illustrating an embodiment of the present disclosure wherein a first sound level 41 is higher at an average of approximately 105 dB, and a second sound level 42 at approximately 93 dB. The sound level may be adapted in strength to impact the infant's ability to hear the output of the audio device 10.

Although the embodiments discussed in this document mainly discusses an audio device 10 emitting white noise at two levels and over two corresponding time periods, it is within the inventive concept that the audio device 10 comprise the feature of outputting many different sound levels, different sound patterns (not only what is categorized as white noise) and different time periods. Such as for example, as illustrated in FIG. 5B, the first time period has a duration of within the mentioned 1-30 seconds at the first sound level 41, but the second time period at the second sound level 42 has a duration of a further limited period 22 until being replaced by a thirds time period 22′, and even further following time periods 22″, and a third, or further, sound level s3 and sound pattern. This may be adapted to the need of the infant, and possibly intelligently analyzed by the audio device 10 to adapt and shift between time periods 31, 32, 32′, 32″ and sound levels s1, s2, s3 according to a programmed, and/or sensed, and/or analyzed, and/or historically experienced, best combination. See FIG. 5B.

The controller 4 controlling the output sound level may comprise features adapting the sound level, duration and pattern in a variety of combinations comprising stepwise and continuous variations of multiple sound levels, durations and patterns. A timer circuit may be provided for maintaining the initial sound level at a constant level for a short period of time, preferably in the range of 1 to 30 seconds. The timer is set to control the second time period, and optionally subsequent time periods, for longer periods, preferably in the range of up to 12 hours or until the audio device 10 is turned off. The audio device 10 may be switched on with a single on/off button and may also be switched off manually with the on/off button after the child has finished sleeping, or other.

In one embodiment of the present invention where the form of the invention is defined, as illustrated in FIG. 1, the components comprised in the audio device 10, is comprised in a housing having the shape of a heart. This special shape of the house is not just an attractive and compact design for placement near the cot or stroller, but it serves the purpose and idea of holding the device safely in the hand or that the device hangs on a string like a necklace around the caretaker's neck down to the heart when they hold and comfort their child. The shape, however, is not locked to be just a heart as it may be provided in other shapes such as ball, moon, star etc.

The first sound level 41 is meant to be a level comparable to that of a crying infant and high enough for it to be able to penetrate the infant's own crying and to attract the babies' attention.

When the duration of the first time period has expired, and observations have shown that a relatively short time period 31 is sufficient, normally a period in the range of up to 5 seconds, the controller 4 regulates audio output to a second sound level 42. The second sound level being sufficiently low to keep the child's attention and to calm him to sleep or at least a state of calm. The required second sound level 42 may be individually defined. Depending on various parameters, from the individual infant's needs, to how close to the infant the audio device 10 is arranged. Nevertheless, the first sound level 41 should be sufficient for the infant to hear it through his own crying, and the second sound level 42 may be lower but high enough for the child to hear it so that the child stays calm.

After a few minutes outputting the second sound level 42, the infant may also fall asleep or enter a state of calm.

In a further embodiment of present invention, the first sound level 41 and/or the second sound level 42, 42′, s3 is adjustable. For this purpose, it may be provided volume buttons 3, 3′ for adjusting output sound level 41, 42, s3. The sound levels may also be instantaneous programmable, such that when manually adjusting the volume when played, the sound level may be stored and remembered to the next time the audio device 10 is used.

It is in one embodiment of the audio device 10 further provided a start button 8 for starting or restarting the audio output sequence 20. This is advantageously used if, for example, the first time period 31 is not sufficient to get the infants attention, it may be possible for a caretaker to restart the first time period 31 by pushing a button 8 provided for this. The restart feature may also be implemented by pushing one or both volume buttons 3, 3′ for a defined time period, for example >3 seconds.

In a further embodiment of present invention the audio device 10 further comprises: a sound sensor 9, and a sound sensor analyzing module 4′. The sound sensor is used to detect ambient sound, such as from the infant himself, or from other sound source. One use of the sound sensor is to detect if the infant is crying, and also the analyzing module 4′ may be used to analyze the crying mode of the infant. As symptoms of colic in infants normally may be differentiated to other types of crying, this may be advantageous to be used by the audio device 10.

The sound sensor analyzing module 4′ may further be enabled to define the first sound level 41 relative the sensed and analyzed sound level from the sound sensor 9. Since the aim is to get the attention of the infant by playing a sound louder than the infants crying, it may be possible to use a lower first sound level 41 for an infant having a lower crying than for some children that may need a higher than average first sound level 41.

The sound sensor analyzing module 4′ may also be used to trigger an automatic restart module 14 for restarting the audio output sequence 20. This may be facilitated for use when the duration of the first time period 31 has expired and the sensed and analyzed sound level from the sound sensor 9 is higher than the second sound level 42. This may indicate that the infant has started crying again, and if the cry shows symptoms of colic behavior pattern, it may be advantageous to attempt outputting a new cycle of a first and a second (further) sound level 41, 42 and time periods 31, 32 as defined above.

The audio device 10 may comprise a power storage device 7 for powering the operation of the device. The power storage device 5,7 may be one of, or a combination of, but not limited to: a one-time use battery 7, a rechargeable battery 7, a capacitor 7, a remote connected power source 5, or a power grid connector 5 connecting the audio device to a power grid.

Since the audio output is normally meant to be operable for hours at end, it is advantageous to use a sufficiently large battery, or a grid connected power connection for powering the device.

For individual adaptability it is also foreseen that the present invention is provided with a configurable audio sound pattern device providing the sound input pattern to be fed to the loudspeaker. For individual purpose it may for example be sufficient, or desirable, to output a predefined sound pattern different than the first white noise sound pattern, such as a human recorded speech/song/sound, and substitute one of the sound pattern 21, 22, 22′, 22″ of the audio device 10 with this predefined sound pattern.

A memory chip 6 inserted in a memory chip reader 6′ may be comprised in the audio device 10 for providing the required audio sound patterns. The memory chip 6 may be removable, and thus different configurable audio sound patterns may be chosen from time to time.

The configurable audio sound pattern device may in further embodiments of present invention be comprising any storage devices usable for storing, playing and/or generating sound pattern/music/noise, such as for example one of, but not limited by: MP3, WAV, FLAG, WMA, MP2, AAC, AC3, AIFF and M4A audio files, and other. Storage may be comprised of any storage medium usable and may be one of but not limited to: RAM, tape, disc, memory chip, USB flash drives, hard or soft coded audio pattern integrated in a circuit board where the circuit board is part of the controller 4 or a dedicated circuit design in communication with the controller 4, and others. It is within the inventive concept that the audio device 10 also comprise reading and executing devices for reading and playing any stored/generated sound pattern.

The audio device 10 may further comprise sensors detecting one or more of, but not limited to: infant movement, infant temperature, heart rhythm, lung/breading rhythm and volume, moisture, damp, smell/odor, and other. Information from such sensors may be used to analyze need for activation and level of output form the audio device 10.

The audio device 10 may further be connected to a remote controller 50, a communication module 51 for communicating with the remote controller 50, and an antenna 52 for signal communication, as illustrated in system configuration in FIG. 7. This is provided for enabling remote configuration and operation of the audio device 10. Remote operation may include switching device on and off, restarting the audio sequence, changing audio sound pattern, defining sound levels and time periods, transmitting sensor output to remote controller and other.

The communication module 51 may be adapted according to any required communication protocol 57, and may thus comprise one or more of, but not limited to: WiFi communication module, Bluetooth communication module, RFID communication module, NFC communication module, Radio Frequency communication module. It may even in some instances be foreseen to provide a wired communication network 58 between the audio device 10 and the remote controller 50. This may for example be in environments where radio communication is restricted or disturbed by other signals.

The remote controller 50 may be one or more of, but not limited to: a smart phone, a tablet, a computer, a cloud based service, or a dedicated communication device. In a family environment it is foreseen that the audio device 10 is paired with a smart phone, for example, a maternity ward at a hospital, multiple audio devices 10 may be communicating with a computer based server application.

Any of the modules and features discussed for the audio device 10 may be facilitated in a comprised custom designed circuit board, and processing resources for handling the controller and communication operation is provided. Although internal design and circuit diagrams are not discussed here, a skilled person is competent to provide such diagrams.

In one embodiment the audio device 10 is adapted for being installed for example on a bedpost of the infant's bed, and then it may be advantageous to provide the audio device 10 with a sound directing device 60 for directing the sound output from the loudspeaker 1. This may be used to direct the sound specifically towards the sleeping infant, and to shield sound propagation in the direction of for example parents laying in a bed beside the infant's bed, such that the parents is not disturbed by output of the device. Even if, for example, a noise level at 70 dB is not harmful, it may be disturbing for a grown person when sleeping, and a directing device 60 may moderate the sound considerably in a selected shielded direction.

Various enclosures for the audio device 10 may be provided, and examples may comprise, but is not limited to: an enclosure 100 for being carried around the neck of a caretaker as exemplified in FIG. 1, and an enclosure 110 for being mounted close to an infant resting pillow/headrest position.

A necklace string with for example a length equal to the minimum advised distance from loudspeaker to nearest infant ear may be provided. The audio device 10 may be provided with a conduit 2 wherein the necklace string may be threaded, such that it readily may be in reach for controlling the distance between the device and the ear of the infant.

The second aspect of this disclosure shows a method for alleviating symptoms of colic in infants, the method comprise the following steps:
providing an audio device 10,
the audio device 10 being provided with the features of:
   outputting an audio white noise output 20 for the duration of a first short period of time 31 louder than an infant's crying first sound level 41,
   when the duration of the first short period of time 31 has expired outputting an noise output:
      a second white noise at a lower sound level 42, or
      a different second noise pattern at a lower sound level 42, and
   keep the second sound level 42 and pattern indefinite or until the infant wakes up and the audio device 10 is turned off manually.

In an advantageous method embodiment of present invention the sound output is provided by an audio device 10 according to the first aspect of this disclosure.

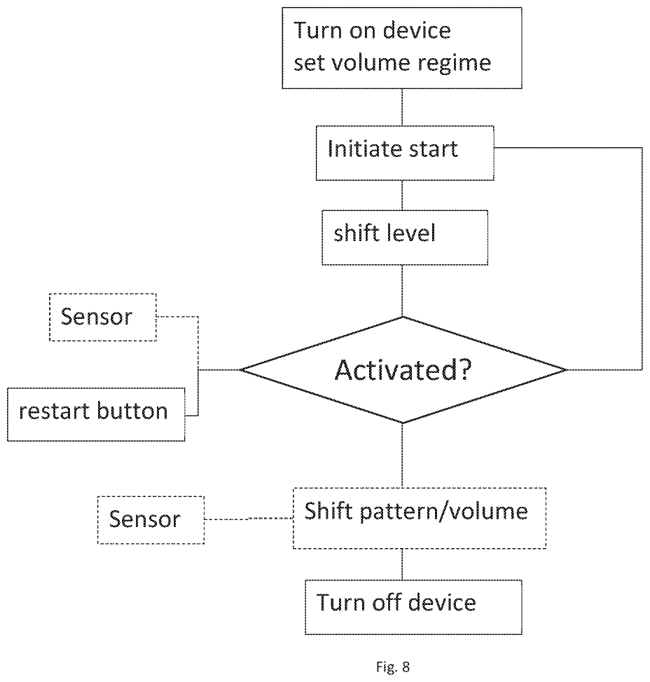

FIG. 8 show one possible method implementation of present invention, where the audio device is first turned on, and optionally adjusted for individual volume setting. When audio output is to start, a start button is pushed. The audio device 10 then starts executing an audio output according to a predefined output regime. After the duration of a predefined first time a shift in audio volume and optional sound pattern changes to a second, and optionally further, level. A restart button is monitored, and when hit the sound pattern output is reinitiated and starts at the beginning. An optional sensor, of any type as discussed, may also be analysed and then activate a restart of output sequence. Further sensor may be used for input to analysis giving reason for audio output adjustments.

All sensors may be connected to the communication modules, or analysed sensor data may be communicated by the communication module, and this way be used for monitoring purposes when sensor data is used in remote controller or remote processing devices.

A third aspect of this disclosure provides a system for alleviating symptoms of colic in infants, the system comprising:
an audio device 10 according to the first aspect of this disclosure, and
a remote processing device 55.

In an advantageous system embodiment of present invention the system further comprises:
a remote controller 50, and
a cloud based service 56 communicating with one or more of the audio device 10, the remote controller 50, and the remote processing device 55.

The remote processing device 55, and or the cloud based services, may comprise record keeping features, custom settings, Artificial Intelligence, AI, services, and other, to adapt the performance of the audio device 10 for best performance fitted to individual needs. Further services may comprise alarm features and broadcasting messages and data to other persons and services.

The audio device 10, and the remote controller 50 and/or remote processing device 55 may also support two way communication mode, such that for example if one sensor is of a microphone type able to monitor sound from the infant surrounding, the monitored sound may be transmitted to the remote controller 50 and/or remote processing device 55. Then a user/caretaker may use the remote controller 50 and/or remote processing device 55 to transmit an oral communication back to the audio device 10, such that the speaker in the audio device 10 may output the oral communication.

Other use of the two way communication feature may be manual observation of sensor data on the remote controller 50 and/or remote processing device 55 side, and use of these input enable an operator/caretaker to manage the sound output regime of the audio device 10.

In a future version of the audio device 10 it is foreseen to implement an on device AI module which may, over time be trained by individual infants, and adapt on-site for best sound patterns, duration of time periods, and volumes, for best performance.

The person skilled in the art realizes that the present disclosure is not limited to the preferred embodiments described above. The person skilled in the art further realizes that modifications and variations are possible within the scope of the appended claims. Additionally, variations to the disclosed embodiments can be understood and effected by the skilled person in practicing the claimed disclosure, from a study of the drawings, the disclosure, and the appended claims.

1. An audio device for alleviating symptoms of colic in infants comprising:
a loudspeaker,
a controller for controlling the loudspeaker,
a sound input pattern to be fed to the loudspeaker, and
a power source,
the audio device is programmable to output audio sequences (20, 20′) at two or more sound levels,
the audio device is programmed to output a first sound pattern during a first time period at a first sound level, and
automatically switch to output a second sound pattern during a second time period at a second sound level when the duration of the first time period has expired, and
the second sound level is lower than the first sound level.

2. (canceled)

3. The audio device according to claim 1, wherein the duration of the first time period is in the region 1-30 seconds, or 1-20 seconds, or 1-10 seconds, or 4-7 seconds, or 5 seconds, with a deviation of +/−0.5 seconds.

4. The audio device according to claim 1, wherein the duration of the second time period is indefinite.

5. (canceled)

6. The audio device according to claim 1, wherein the first sound level and/or the second sound level is adjustable.

7. The audio device according to claim 1, the audio device further comprises a start button for starting or restarting the audio output sequence.

8. The audio device according to claim 1, the audio device further comprises:
a sound sensor, and
a sound sensor analyzing module wherein the sound sensor analyzing module is enabled to define the first sound level relative the sensed and analyzed sound level from the sound sensor.

9. (canceled)

10. The audio device according to according to claim 8, the audio device further comprises an automatic restart module for restarting the audio output sequence when the duration of the first time period has expired and sensed and analyzed sound level from the sound sensor is higher than the second sound level.

11. The audio device according to claim 1, further comprising a power storage device for powering the audio device, wherein the power storage device is one of, or a combination of: a one-time use battery a rechargeable battery a capacitor a remote connected power source or a power grid connector connecting the audio device to a power grid.

12. (canceled)

13. The audio device according to claim 1, further comprising a configurable audio sound pattern device providing the sound input pattern to be fed to the loudspeaker.

14. The audio device according to claim 13, wherein
the configurable audio sound pattern device is a memory chip inserted in a memory chip reader comprised in the audio device.

15. The audio device according to claim 1, further comprising:
a remote controller,
a communication module for communicating with the remote controller, and
an antenna for signal communication.

16. The audio device according to claim 15, wherein the communication module comprise one or more of: Wi-Fi communication module, Bluetooth communication module, RFID communication module, NFC communication module, Radio Frequency communication module.

17. The audio device according to claim 15, wherein the remote controller is one or more of: a smart phone, a tablet, a computer, a cloud based service, or a dedicated communication device.

18. The audio device according to claim 1, further comprising a sound directing device for directing the sound output from the loudspeaker.

19. The audio device according to claim 1, wherein the audio device is comprised in one of:
an enclosure for being carried around the neck of a caretaker, and
an enclosure for being mounted close to an infant resting pillow/headrest position.

20. The audio device according to claim 19, wherein a necklace string with a length equal to minimum distance from loudspeaker to nearest infant ear.

21. (canceled)

22. A method for alleviating symptoms of colic in infants, the method comprise the following steps:
providing an audio device according to claim 1,
the audio device being provided with the features of:
outputting an audio white noise for the duration of a first short period of time louder than an infant's crying first sound level,
when the duration of the first short period of time has expired outputting:
a second white noise at a lower sound level, or
a different second noise pattern at a lower sound level, and
keep the second sound level and pattern indefinite or until the infant wakes up.

23. (canceled)

24. A system for alleviating symptoms of colic in infants, the system comprising:
an audio device according to claim 1, and
a remote processing device.

25. The system according to claim 24, further comprising:
remote controller, and
a cloud based service communicating with one or more of the audio device, the remote controller, and the remote processing device (55).
